(19) United States
(12) Patent Application Publication
(10) Pub. No.: US 2025/0303151 A1
(43) Pub. Date: Oct. 2, 2025

(54) Applying Alternating Electric Fields to a Subject's Body in Multiple Directions, with Certain Directions Being Prioritized

(71) Applicant: Novocure GmbH, Baar (CH)

(72) Inventors: Ariel NAVEH, Haifa (IL); Ari M. FRANK, Haifa (IL)

(73) Assignee: Novocure GmbH, Baar (CH)

(21) Appl. No.: 19/093,569

(22) Filed: Mar. 28, 2025

Related U.S. Application Data
(60) Provisional application No. 63/571,547, filed on Mar. 29, 2024.

(51) Int. Cl. A61N 1/32; A61N 1/08

(52) U.S. Cl. CPC A61N 1/32; A61N 1/08

(57) ABSTRACT

Alternating electric fields (e.g., TTFields) are applied in a region of interest (ROI) by (a) inducing a first electric field in a first direction in the ROI for a first duration of time T1; (b) inducing a second electric field in a second direction in the ROI for a second duration of time T2; and repeating step (a) and step (b) in an alternating sequence. If a determination has previously been made that the first electric field will provide a larger therapeutic effect than the second electric field, the durations T1 and T2 are selected so that, in the aggregate, step (a) is performed for more time than step (b). This increases the overall therapeutic effect of the TTFields with respect to systems that do not prioritize one of the directions over the other direction.

### CROSS REFERENCE TO RELATED APPLICATIONS

This Application claims the benefit of U.S. Provisional Application 63/571,547, filed Mar. 29, 2024, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 50 kHz-1 MHz, 50-500 kHz, 75-300 kHz, or 150-250 kHz). FIG. 1 depicts the prior art Optune® system, which delivers 200 kHz TTFields to patients via four electrode assemblies (also referred to as “transducer arrays”) that are placed on the patient's skin near the tumor. In the version of Optune® that is used to treat glioblastoma, each transducer array includes nine electrode elements. The transducer arrays are arranged in two pairs (or “channels”), with one pair of transducer arrays 10L, 10R positioned to the left and right of the tumor, and the other pair of transducer arrays 10A, 10P positioned anterior and posterior to the tumor. Each transducer array is connected via a multi-wire cable to an AC signal generator 15.

Optune's AC signal generator (a) sends an AC current through the anterior/posterior (A/P) pair of transducer arrays for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment. Accordingly, both the L/R channel and the A/P channel operate at a 50% duty cycle, as depicted in FIG. 2.

Increasing the amplitude of Optune's output will typically yield a corresponding increase in efficacy. But because higher amplitudes also cause the transducer arrays to heat up, the amplitude usually cannot be dialed up to its maximum possible setting. Instead, temperature sensors are incorporated into Optune's transducer arrays, and the Optune® system is configured to slowly increase its output voltage until the hottest transducer array reaches a safety temperature threshold (e.g., 39° C.). At this point, Optune® stops increasing its output voltage.

### SUMMARY OF THE INVENTION

One aspect of the invention is directed to a first apparatus for applying alternating electric fields to a region of interest. The first apparatus comprises a signal generator having a first output, a second output, and at least one data input. The first output is configured to apply a first 50 kHz-1 MHz alternating voltage between a first electrode assembly and a second electrode assembly, and the second output is configured to apply a second 50 kHz-1 MHz alternating voltage between a third electrode assembly and a fourth electrode assembly. The at least one data input is configured to accept data that represents temperatures of the first, second, third, and fourth electrode assemblies. The signal generator is configured to repeatedly activate the first and second outputs in an alternating sequence. The signal generator is also configured to adjust how long the first and second outputs are activated based on data that represents temperatures of the first and second electrode assemblies, so that a duty cycle of the first output is 85-100% of a largest duty cycle that prevents the first and second electrode assemblies from exceeding a temperature threshold Tmax. And the signal generator is configured to only activate the second output when the first output is deactivated.

In some embodiments of the first apparatus, the duty cycle of the first output is 95-100% of the largest duty cycle that prevents the first and second electrode assemblies from exceeding the temperature threshold Tmax. In some embodiments of the first apparatus, the duty cycle of the first output is 98-100% of the largest duty cycle that prevents the first and second electrode assemblies from exceeding the temperature threshold Tmax.

Some embodiments of the first apparatus further comprise the first electrode assembly, the second electrode assembly, the third electrode assembly, and the fourth electrode assembly.

In some embodiments of the first apparatus, the signal generator has a third output configured to apply a third 50 kHz-1 MHz alternating voltage between a fifth electrode assembly and a sixth electrode assembly, and the at least one data input is configured to accept data that represents temperatures of the fifth and sixth electrode assemblies. The signal generator is configured to repeatedly activate the first, second, and third outputs in an alternating sequence. The signal generator is configured to adjust how long the first, second, and third outputs are activated based on data that represents temperatures of the first, second, third, and fourth electrode assemblies. And the signal generator is configured to only activate the second and third outputs when the first output is deactivated. Optionally, in these embodiments, the signal generator can be configured to only activate the third output when the first and second outputs are deactivated.

Another aspect of the invention is directed to a first method of applying alternating electric fields to a region of interest. The first method comprises (a) applying a 50 kHz-1 MHz alternating voltage between a first electrode assembly and a second electrode assembly positioned at respective first and second locations on opposite sides of the region of interest, so that a first electric field is induced in the region of interest; (b) applying a 50 kHz - 1 MHz alternating voltage between a third electrode assembly and a fourth electrode assembly positioned at respective third and fourth locations on opposite sides of the region of interest, so that a second electric field is induced in the region of interest; and (c) repeating step (a) and step (b) in an alternating sequence at least 100 times. Step (a) and step (b) are performed with respective durations so that a duty cycle of step (a) is 85-100% of a largest duty cycle that prevents the first and second electrode assemblies from exceeding a temperature threshold Tmax.

In some instances of the first method, the duty cycle of step (a) is 95-100% of the largest duty cycle that prevents the first and second electrode assemblies from exceeding the temperature threshold Tmax. In some instances of the first method, the duty cycle of step (a) is 98-100% of the largest duty cycle that prevents the first and second electrode assemblies from exceeding the temperature threshold Tmax.

Some instances of the first method further comprise running a plurality of simulations, each of which involves (a) positioning a plurality of model electrode assemblies at respective locations on a model of a subject's body and (b) predicting a therapeutic effect that a resulting electric field will provide within the region of interest when an alternating voltage is applied between the plurality of model electrode assemblies; and selecting the first and second locations based on the plurality of simulations. The first and second locations correspond to locations of the model electrode assemblies that provided the largest of all the predicted therapeutic effects.

Some instances of the first method further comprise running a plurality of simulations, each of which involves (a) positioning a plurality of model electrode assemblies at respective locations on a model of a subject's body and (b) predicting a therapeutic effect that a resulting electric field will provide within the region of interest when an alternating voltage is applied between the plurality of model electrode assemblies; and selecting the first and second locations based on the plurality of simulations. The first and second locations correspond to locations of the model electrode assemblies that provided a predicted therapeutic effect that was within the top 10 percent of all the predicted therapeutic effects. Optionally, these embodiments may further comprise selecting the third and fourth locations based on the selected first and second locations.

Another aspect of the invention is directed to a second method of applying alternating electric fields to a region of interest. The second method comprises (a) applying a 50 kHz-1 MHz alternating voltage between a first electrode assembly and a second electrode assembly positioned at respective first and second locations on opposite sides of the region of interest, so that a first electric field is induced in the region of interest; (b) applying a 50 kHz-1 MHz alternating voltage between a third electrode assembly and a fourth electrode assembly positioned at respective third and fourth locations on opposite sides of the region of interest, so that a second electric field is induced in the region of interest; and repeating step (a) and step (b) in an alternating sequence at least 100 times. Step (a) and step (b) are performed with respective durations so that either (i) in the aggregate, step (a) is performed for more time than step (b) if a determination has previously been made that the first electric field will provide a larger therapeutic effect than the second electric field, or (ii) in the aggregate, step (b) is performed for more time than step (a) if a determination has previously been made that the second electric field will provide a larger therapeutic effect than the first electric field.

Some instances of the second method further comprise (c) applying a 50 kHz-1 MHz alternating voltage between a fifth electrode assembly and a sixth electrode assembly positioned at respective fifth and sixth locations on opposite sides of the region of interest, so that a third electric field is induced in the region of interest. In these instances, step (a), step (b), and step (c) are repeated in an alternating sequence at least 100 times, and step (a) is performed for more time than step (b) and for more time than step (c) if a determination has previously been made that the first electric field will provide a larger therapeutic effect than the second electric field and the third electric field.

Some instances of the second method further comprise determining whether the first electric field or the second electric field will provide a larger therapeutic effect. The determining occurs before the repeating.

Some instances of the second method further comprise simulating the first electric field in the region of interest based on (i) the respective locations of the first and second electrode assemblies and (ii) characteristics of tissue located between the first and second electrode assemblies; simulating the second electric field in the region of interest based on (i) the respective locations of the third and fourth electrode assemblies and (ii) characteristics of tissue located between the third and fourth electrode assemblies; and determining whether the first electric field or the second electric field will provide a larger therapeutic effect by comparing the simulation of the first electric field to the simulation of the second electric field. The determining occurs before the repeating.

Optionally, in the instances described in the previous paragraph, the comparing of the simulations comprises comparing field strengths within the region of interest for the first electric field to field strengths within the region of interest for the second electric field. Optionally, in the instances described in the previous paragraph, the comparing of the simulations comprises comparing power densities within the region of interest for the first electric field to power densities within the region of interest for the second electric field.

In some instances of the second method, if the determination has previously been made that the first electric field will provide a larger therapeutic effect than the second electric field, step (a) and step (b) are performed with respective durations so that, in the aggregate, step (a) is performed for at least 10% more time than step (b). In some instances of the second method, if the determination has previously been made that the first electric field will provide a larger therapeutic effect than the second electric field, step (a) and step (b) are performed with respective durations so that, in the aggregate, step (a) is performed for at least 25% more time than step (b). In some instances of the second method, if the determination has previously been made that the first electric field will provide a larger therapeutic effect than the second electric field, step (a) and step (b) are performed with respective durations so that the time spent performing step (a) is maximized to an extent that is possible in view of thermal considerations, and so that step (b) is performed as needed to prevent the first electrode assembly and the second electrode assembly from exceeding a temperature threshold.

In some instances of the second method, the first electric field and the second electric field are perpendicular, ±30°, within the region of interest.

### BRIEF DESCRIPTION OF THE DRAWINGS

FIG. 1 is a block diagram of a prior art system for delivering TTFields to a subject's body using a set of electrode assemblies.

FIG. 2 is a timing diagram associated with the FIG. 1 prior art system.

FIG. 3 is a block diagram of a two-channel system for delivering alternating electric fields to a subject's body using a set of electrode assemblies, in which one of the channels is prioritized.

FIG. 4 is a timing diagram associated with the FIG. 3 embodiment.

FIG. 5 is a flowchart of one approach for favoring the prioritized channel that can run on the hardware depicted in FIG. 3.

FIG. 6 is a flowchart of another approach for favoring the prioritized channel that can run on the hardware depicted in FIG. 3.

Various embodiments are described in detail below with reference to the accompanying drawings, wherein like reference numerals represent like elements.

### DESCRIPTION OF THE PREFERRED EMBODIMENTS

Notably, the prior art Optune® system described above always activates the A/P channel for the same amount of time that it activates the L/R channel, and it does not exhibit a preference for either one of those channels; and this “indifferent” approach works well in most anatomic situations. But in certain anatomic situations (or in certain individual subjects), it may turn out that one of the channels provides a larger therapeutic effect than the other channel. This could occur, for example, when both channels are operating at the same voltage, but the field strength that is induced by one of the channels in a region of interest (ROI) is significantly higher than the field strength that is induced by the other channel in the same ROI. (Higher field strengths provide larger therapeutic effects.) When these situations arise, it can be beneficial to activate the channel that provides the larger therapeutic effect for a larger proportion of time than the channel that provides the smaller therapeutic effect.

The embodiments described below can take advantage of these situations by designating one of the channels (e.g., the channel that provides the larger therapeutic effect) as the “preferred” channel and designating the other channel (e.g., the channel that provides the smaller therapeutic effect) as the “non-preferred” channel, and activating the preferred channel for a larger proportion of time than the non-preferred channel. The decision as to which channel should be designated as being the preferred channel can be based on simulations that compute the respective field strengths (or power densities) that the A/P channel and the L/R channel will provide in the ROI, and designating the channel that has the higher field strength as the preferred channel. Alternatively, the decision as to which channel should be designated as being the preferred channel can be based on factors that do not rely on simulations.

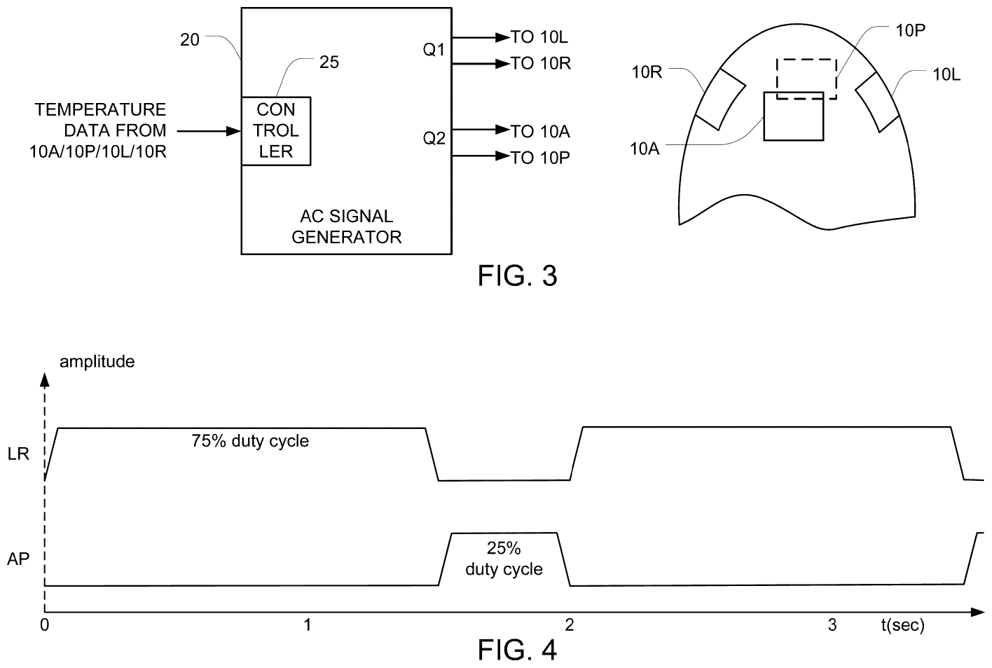

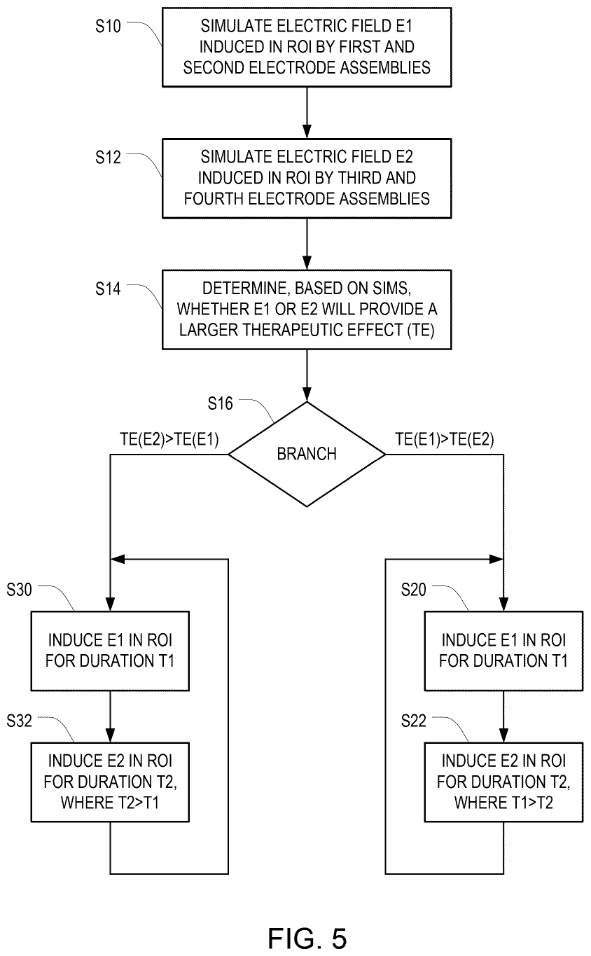

FIG. 3 is a block diagram of an embodiment that activates the preferred channel for a larger proportion of time than the non-preferred channel, FIG. 4 is an example of a timing diagram that depicts the amount of time each channel is activated, and FIG. 5 is a flowchart of one approach for favoring a preferred channel that can run on the hardware depicted in FIG. 3.

Turning first to FIG. 3, this embodiment relies on electrode assemblies 10L/10R/10A/10P that are positioned on the subject's body. These electrode assemblies can be similar to the electrode assemblies that are used in the prior art Optune® system, or can be constructed using another approach, e.g., any of the approaches described in US 2023/0043071, US 2021/0402179, and U.S. Pat. No. 8,715,203, each of which is incorporated herein by reference in its entirety.

As in the prior art Optune® system, the electrode assemblies are arranged in two pairs (or “channels”), with one pair of electrode assemblies 10L, 10R positioned in or on the subject's body to the left and right of the ROI, and the other pair of electrode assemblies 10A, 10P positioned in or on the subject's body anterior and posterior to the ROI. Thus, the first and second electrode assemblies 10L and 10R are positioned at respective first and second locations on opposite sides of the ROI, and the third and fourth second electrode assemblies 10A and 10P are positioned at respective third and fourth locations on opposite sides of the ROI. Each electrode assembly is connected via a multi-wire cable to The AC signal generator 20.

The AC signal generator 20 has first and second outputs Q1 and Q2, and a data input. Each of the outputs Q1 and Q2 operates between 50 kHz and 1 MHz (e.g., 50-500 kHz, 75-300 kHz, or 150-250 kHz). In the embodiment illustrated in FIG. 3, the first output Q1 applies an alternating voltage between the first and second electrode assemblies 10L/10R, and the second output Q2 applies an alternating voltage between the third and fourth electrode assemblies 10A/10P. But in alternative embodiments (not shown) each of those outputs Q1, Q2 could drive more than two electrode assemblies. This could occur, for example, by having the first output Q1 apply an alternating voltage between (a) two or more first electrode assemblies on the left side of the subject's body and (b) two or more second electrode assemblies on the right side of the subject's body. In another example, the second output Q2 could apply an alternating voltage between (a) a single third electrode assembly on the anterior side of the subject's body and (b) two or more fourth electrode assemblies on the posterior side of the subject's body. In other alternative embodiments (not shown) there can be one or more additional outputs (e.g., Q3, Q4, etc.), each of which drives at least one additional pair of electrode assemblies (not shown).

One way to implement the AC signal generator 20 is to use the hardware described in U.S. Pat. No. 9,910,453 (which is incorporated herein by reference in its entirety), but with modifications that enable the AC signal generator 20 to perform the functions and operations described herein. Alternatively, a variety of alternative approaches for implementing the AC signal generator 20 that will be apparent to persons skilled in the relevant arts can also be used.

The AC signal generator 20 incorporates a controller 25, and the controller 25 issues commands to which the rest of the AC signal generator 20 responds e.g., as described below. Note that while the controller 25 and the AC signal generator 20 are depicted as being integrated into a single device 20 in FIG. 3, those two blocks 20, 25 could be implemented as two discrete hardware devices that are connected by appropriate cabling. Furthermore, the functions and operations that are described herein as being performed by the AC signal generator 20 could be distributed amongst a plurality of discrete hardware devices that are connected by appropriate cabling. For example, instead of using an AC signal generator that has two outputs, only one of which is active at any given time, an AC generator that has a single output that is always on can be used, followed by a switch that routes that single output to either the L/R or the A/P electrode assemblies. In this case, the combination of the single-output AC generator and the switch would collectively replace the AC signal generator 20 described herein (in which case those two components would collectively serve as the AC signal generator 20 described herein).

The AC signal generator 20 also has at least one data input configured to accept data that represents temperatures of the first, second, third, and fourth electrode assemblies 10. In some embodiments, thermistors are incorporated into the electrode assemblies 10 to sense the temperature of the electrode assemblies 10, in which case the AC signal generator 20 includes hardware configured to accept signals from those thermistors so that the controller 25 can make decisions based on the temperature. In other embodiments, other temperature sensors (e.g., RTDs or integrated circuit temperature sensors such as the Texas Instruments TMP1075DSGR) can be used, in which case the AC signal generator 20 should include hardware configured to accept signals from whatever temperature sensors are used.

The AC signal generator 20 (a) activates the first output Q1, which applies an alternating voltage between the first and second electrode assemblies 10L/10R for a duration of time T1, which induces a first electric field E1 in the ROI, then (b) activates the second output Q2, which applies an alternating voltage between the third and fourth electrode assemblies 10A/10P for a duration of time T2, which induces a second electric field E2 in the ROI. In some preferred embodiments, the electrode assemblies 10 are positioned so that first electric field and the second electric field are perpendicular, ±30, within the ROI. The AC signal generator 20 then repeats step (a) and step (b) in an alternating sequence. The alternating sequence can be repeated, for example, at least 100 times.

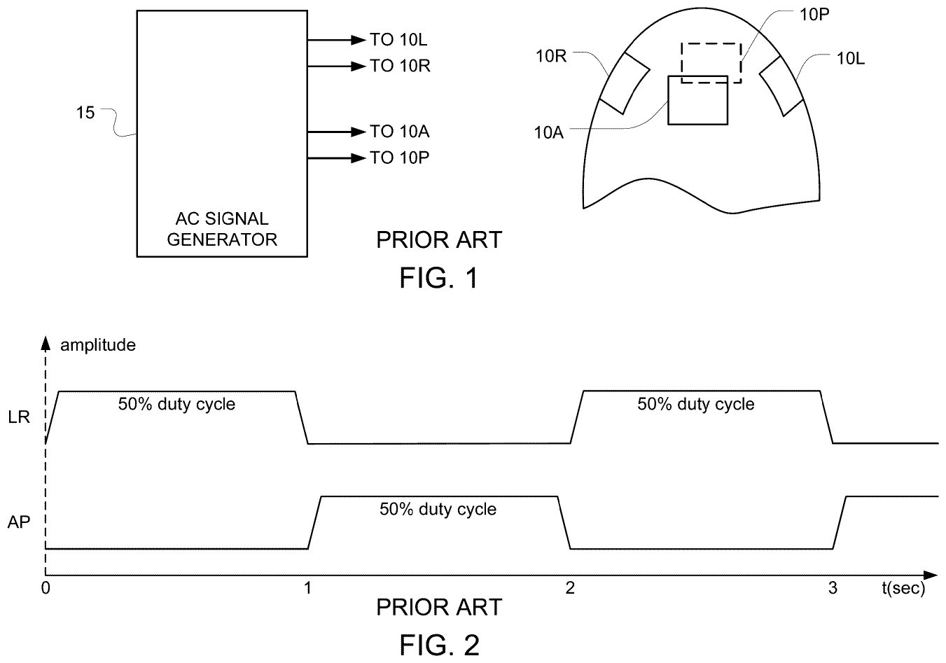

Notably, unlike the prior art Optune® system depicted in FIGS. 1 and 2 in which the durations of time T1 and T2 are the same, the durations T1 and T2 are selected so that the preferred channel (i.e., the channel that provides the larger therapeutic effect) remains active for more time than the non-preferred channel.

In the example depicted in FIG. 4, the L/R channel remains active for more time than the A/P channel. More specifically, the L/R channel remains on for 1.5 seconds and off for 0.5 seconds in this example (which corresponds to a 75% duty cycle) while the A/P channel remains on for 0.5 seconds and off for 1.5 seconds (which corresponds to a 25% duty cycle). (Note that this example is based on the assumption that the L/R channel has previously been identified as a channel that will provide the larger therapeutic effect. As a result, the L/R channel is the preferred channel in this example.) And because the channel that provides the larger therapeutic effect is active for more time than the non-preferred channel (and has a corresponding higher duty cycle), the overall therapeutic effect will be better than the overall therapeutic effect that was obtained using the prior art Optune® system.

FIG. 5 is a flowchart depicting a set of steps that can be initiated by the controller 25 depicted in FIG. 3 to implement this process. First, in S10, a simulation is run to quantify the first electric field E1 that will be induced in the ROI when an alternating voltage is applied between the first and second electrode assemblies (i.e., the L/R electrode assemblies). Then, in S12, a simulation is run to quantify the second electric field E2 that will be induced in the ROI when an alternating voltage is applied between the third and fourth electrode assemblies (i.e., the A/P electrode assemblies).

In some embodiments, the simulations referenced in S10 and S12 simulate the first electric field E1 in the ROI based on (i) the respective locations of the first and second electrode assemblies and (ii) characteristics of tissue located between the first and second electrode assemblies, and simulate the second electric field E2 in the ROI based on (i) the respective locations of the third and fourth electrode assemblies and (ii) characteristics of tissue located between the third and fourth electrode assemblies.

Next, in S14, the results of these two simulations are compared to ascertain whether the first electric field E1 or the second electric field E2 will provide a larger therapeutic effect. This comparing could comprise comparing field strengths within the ROI for the first electric field E1 to field strengths within the ROI for the second electric field E2 and/or comparing power densities within the ROI for the first electric field E1 to power densities within the ROI for the second electric field E2.

Note that running simulations (as described above in connection with S10-S14) is not the only way to ascertain whether the first electric field E1 or the second electric field E2 will provide a larger therapeutic effect, and other approaches may be used to make that decision. For example, a given channel can be designated as the preferred channel based on general anatomic features, anatomic features that are specific to the individual subject being treated, and/or data that has been compiled from a population of subjects.

S16 is a branching operation in which processing proceeds to the right if it has previously been determined that the therapeutic effect of the first electric field E1 is greater than the therapeutic effect of the second electric field E2, or proceeds to the left if it has previously been determined that the therapeutic effect of the second electric field E2 is greater than the therapeutic effect of the first electric field E1.

If it has been determined that the therapeutic effect of the first electric field E1 is greater than the therapeutic effect of the second electric field E2, processing continues at S20, during which the first electric field E1 is induced in the ROI for a duration T1. Processing then proceeds to S22 during which the second electric field E2 is induced in the ROI for a duration T2. S20 and S22 are then repeated in an alternating sequence for the duration of the treatment, with the durations of T1 and T2 set so that T1>T2. As a result, in the aggregate, S20 will be performed for more time (e.g., at least 5% more time, at least 10% more time, at least 25% more time, or at least 50% more time) than S22.

On the other hand, if it has been determined that the therapeutic effect of the second electric field E2 is greater than the therapeutic effect of the first electric field E1, processing continues at S30, during which the first electric field E1 is induced in the ROI for a duration T1. Processing then proceeds to S32 during which the second electric field E2 is induced in the ROI for a duration T2. S30 and S32 are then repeated in an alternating sequence for the duration of the treatment, with the durations of T1 and T2 set so that T2>T1. As a result, in the aggregate, S32 will be performed for more time (e.g., at least 5% more time, at least 10% more time, at least 25% more time, or at least 50% more time) than S30.

When it is known in advance which channel will provide the larger therapeutic effect, the branching decision at S16 and either the left or right branch can be omitted. For example, if it is known in advance that the first electric field E1 will provide a larger therapeutic effect than the second electric field E2, steps S16, S30, and S32 can be omitted, in which case processing will always proceed directly to step S20 and S22.

Returning to FIG. 3, in some embodiments the output voltages at the outputs Q1 and Q2 of the AC signal generator 20 are always linked, so that those output voltages always rise or fall in tandem. In these embodiments, when both the L/R channel and the A/P channel operate at the same duty cycle (as in the prior art Optune® system), if a single one of the electrode assemblies 10L/10R/10A/10P approaches the safety temperature threshold (e.g., 39° C.) before the other electrode assemblies 10, then the channel that includes that single (i.e., the hottest) electrode assembly would become a limiting factor from a thermal perspective. If the hottest electrode assembly happens to reside in the non-preferred channel, increasing the amount of time that the preferred channel is on (as described herein) will therefore provide two benefits simultaneously. First, because the preferred channel will be operating for more time, the overall therapeutic effect of the alternating electric fields will increase. And second, because the nonpreferred channel will be operating for less time, the temperature at the hottest electrode assembly will drop, which will ameliorate the thermal limitation. If, on the other hand, the hottest electrode assembly happens to reside in the preferred channel, the amount of time that the preferred channel is on can only be increased in these embodiments if the output voltages of both Q1 and Q2 are decreased.

In other embodiments, the output voltages at the outputs Q1 and Q2 of the AC signal generator 20 can be adjusted independently. In these embodiments, if the hottest electrode assembly happens to reside in the non-preferred channel, increasing the amount of time that the preferred channel is on (as described herein) will provide the same two benefits described in the previous paragraph. But if the hottest electrode assembly happens to reside in the preferred channel, the AC signal generator 20 can reduce the output voltage of the preferred channel only (which will ameliorate the thermal limitation) and simultaneously increase the amount of time that the preferred channel is on. And because the preferred channel will be operating for more time, the overall therapeutic effect of the alternating electric fields should increase.

The example described above in connection with FIG. 3-5 assumes that two channels are used to apply the alternating electric fields in two directions, and that there is a single preferred channel and a single non-preferred channel. But in alternative embodiments, there can be more than one nonpreferred channel. For example, two additional electrode assemblies (i.e., a fifth electrode assembly and a sixth electrode assembly) can be positioned at respective fifth and sixth locations on opposite sides of the ROI, so that a third electric field E3 will be induced in the ROI when an alternating voltage is applied between the fifth and sixth electrode assemblies. In this situation, the AC signal generator 20 can be configured to (a) apply an alternating voltage between the first and second electrode assemblies 10L/10R for a duration of time T1, which induces a first electric field E1 in the ROI, then (b) apply an alternating voltage between the third and fourth electrode assemblies 10A/10P for a duration of time T2, which induces a second electric field E2 in the ROI, and the (c) apply an alternating voltage between the fifth and sixth electrode assemblies (not shown) for a duration of time T3, which induces a third electric field E3 in the ROI. Step (a), step (b), and step (c) are then repeated in an alternating sequence at least 100 times for the duration of the treatment. If a determination has previously been made that the first electric field E1 will provide a larger therapeutic effect than the second electric field E2 and the third electric field E3, then step (a) is performed for more time than step (b) and for more time than step (c). In this situation, the durations T1, T2, and T3 are selected so that T1>T2 and T1>T3. As a result, the first channel (i.e., the channel that provides the larger therapeutic effect) will remain active for more time than both of the non-preferred channels (i.e., the second and third channels).

The embodiments described above in connection with FIGS. 3-5 favor the preferred channel by ensuring that the preferred channel operates for more time than the non-preferred channel. For example, if a determination has been made that the first channel (i.e., the L/R channel in the example above) is the preferred channel, the embodiments described above will favor the preferred channel by setting T1 and T2 so that T1>T2. Therefore, in the aggregate, the first channel will be active for more time than the second channel.

But other approaches for favoring the preferred channel may also be implemented. For example, the AC signal generator can adjust how long the first and second outputs are activated based on data that represents temperatures of the electrode assemblies, so that the first output Q1 remains active for as much time as possible without allowing any of the electrode assemblies to exceed a temperature threshold.

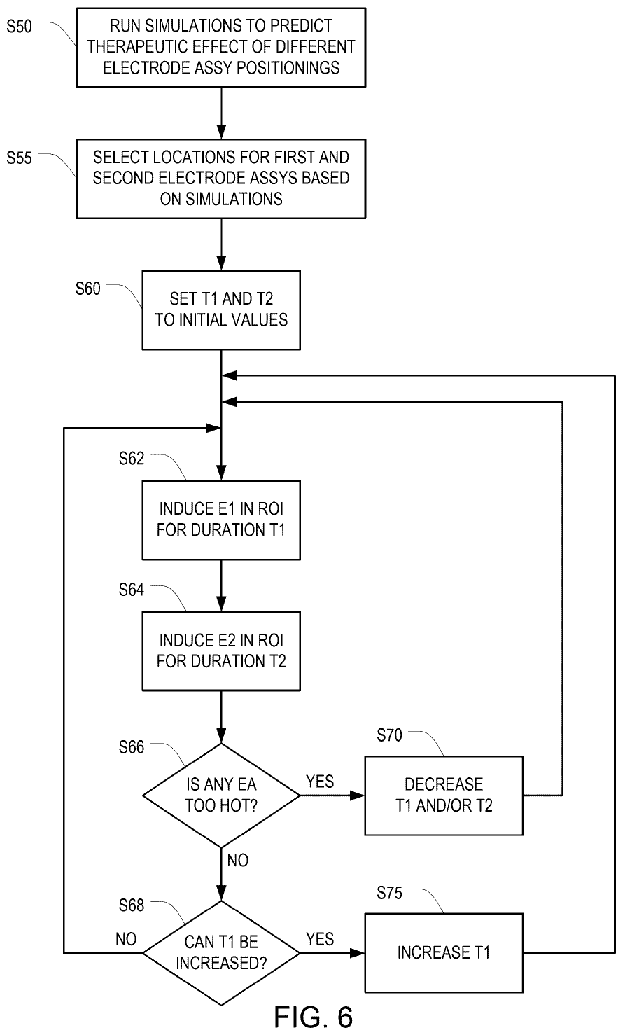

An example of such an approach is depicted in FIG. 6, which is a flowchart of a second approach for favoring a preferred channel. This approach uses the same hardware depicted in FIG. 3, except that the controller 25 controls the operation of the AC signal generator 20 in a different way as described below. Each of the electrode assemblies 10 depicted in FIG. 3 includes one or more temperature sensors, and data from these temperature sensors is routed to the controller 25 so that the controller 25 can make appropriate temperature-based decisions, as described below in connection with FIG. 6.

The FIG. 6 process begins at S50 in which a plurality of simulations are run. Each of the simulations involves (a) positioning two model electrode assemblies at respective locations on a model of a subject's body so that the model electrodes are on opposite sides of the ROI and (b) predicting a therapeutic effect that a resulting electric field will provide within the ROI when an alternating voltage is applied between the two model electrode assemblies. The prediction of the therapeutic effect could be based on a variety of criteria including but not limited to the average intensity of the electric field in the ROI, the average power density in the ROI, the percentage of the ROI that has a field strength above a given threshold (e.g., 1 V/cm, 2 V/cm, etc.), and the percentage of the ROI that has a power density above a given threshold.

Next, in S55, respective locations for the first and second electrode assemblies are selected that will provide the largest of all the predicted therapeutic effects (based on the simulations), and the first and second electrodes are positioned at those locations. In alternative embodiments, instead of selecting the locations that will provide the absolute largest of all the predicted therapeutic effects, locations that provide therapeutic effects within the top 10 percent of all the predicted therapeutic effects can be used. Because these locations provide the best (or near best) therapeutic effects, the channel that corresponds to the first and second electrode assemblies (and the first electric field E1) is designated as the preferred channel.

Another set of electrode assemblies (i.e., third and fourth electrode assemblies) are positioned at respective third and fourth locations on opposite sides of the ROI. The channel that corresponds to the third and fourth electrode assemblies (and the second electric field E2) is designated as the non-preferred channel. In some embodiments, the third and fourth locations are selected based on the previously selected first and second locations (e.g., to prevent the third and fourth electrode assemblies from overlapping any portion of the first and second electrode assemblies, or to provide the highest therapeutic effect possible when constrained to the portion of the subject's body that remains vacant after the first and second electrode assemblies have been positioned).

Note that running simulations (as described above in connection with S50-S55) is not the only way to ascertain where to position the first and second electrode assemblies that correspond to the preferred channel, and other approaches may be used to make that decision. For example, a given channel can be designated as the preferred channel based on general anatomic features, anatomic features that are specific to the individual subject being treated, and/or data that has been compiled from a population of subjects.

The controller 25 controls the AC signal generator 20 so that the AC signal generator will (a) activate the first output Q1, which will apply an alternating voltage between the first and second electrode assemblies, which induces a first electric field E1 in the ROI; and then (b) activate the second output Q2, which will apply an alternating voltage between the third and fourth electrode assemblies, which induces a second electric field E2 in the ROI. The AC signal generator 20 then repeats step (a) and step (b) in an alternating sequence (e.g., at least 100 times). In some embodiments, the AC signal generator 20 is configured to adjust how long the first and second outputs Q1 and Q2 are activated based on data that represents temperatures of the first and second electrode assemblies, so that a duty cycle of step (a) is the largest duty cycle that prevents the first and second electrode assemblies from exceeding a temperature threshold Tmax. In other embodiments, AC signal generator 20 adjusts the outputs Q1 and Q2 so that the duty cycle of step (a) is 85-100%, 90-100%, 95-100%, 98-100%, or 99-100% of the largest duty cycle that prevents the first and second electrode assemblies from exceeding the temperature threshold Tmax. Note that the first and second outputs Q1 and Q2 are never both activated at the same time.

The concept of the largest duty cycle that prevents the electrode assemblies from exceeding a temperature threshold Tmax will now be explained using an example. The effectiveness of alternating electric fields increases when the field strength is stronger, and when the field is applied for longer durations of time. But the field strength and duration cannot be arbitrarily increased because the electrode assemblies heat up during use, and the temperature of the electrode assemblies must be kept below a safety temperature threshold Tmax (e.g., 39° C.) for safety reasons.

Two important factors that contribute to the temperature rise of any given electrode assembly are the current that is being driven through the electrode assembly and the percentage of time that the electrode assembly is active (i.e., the duty cycle). Assume, for example, that a given electrode assembly within the preferred channel will reach Tmax if a given current (e.g., 1 A) is driven through the electrode assembly at a duty cycle of 75% (which is the situation depicted in FIG. 4 for the L/R channel). Because the preferred channel provides a larger therapeutic effect than the non-preferred channel, selecting a duty cycle of 75% for the preferred channel will provide the biggest improvement in the overall therapeutic effect (as compared to the prior art Optune® system depicted in FIG. 1-2, which always uses a 50% duty cycle for both the L/R and A/P channels). Note that in this example, the duty cycle of the preferred channel cannot be increased beyond 75% because that would cause the temperature of one of the electrode assemblies within the preferred channel to exceed Tmax. Note also that when the duty cycle of the preferred channel is increased to 75%, the duty cycle of the nonpreferred channel necessarily decreases to 25%.

While increasing the duty cycle all the way to 75% (i.e., the largest duty cycle that prevents the first and second electrode assemblies from exceeding Tmax in this example) will provide the biggest possible improvement, smaller increases in the duty cycle will still provide a benefit (albeit a smaller benefit). For example, increasing the duty cycle to 85% of the largest duty cycle that prevents the first and second electrode assemblies from exceeding Tmax (i.e., 0.85×75%=a 63.75% duty cycle) will still provide an improvement with respect to the prior art Optune® system, and increasing the duty cycle to 95% or 98% of the largest duty cycle that prevents the first and second electrode assemblies from exceeding Tmax (i.e., 0.95×75%=a 71.25% duty cycle or 0.98×75%=a 73.5% duty cycle) will provide successively larger levels of improvement.

In the preceding example, 75% is the largest duty cycle that prevents the first and second electrode assemblies from exceeding Tmax. But depending on a variety of factors, the largest duty cycle that prevents the first and second electrode assemblies from exceeding Tmax can vary between 50% and 100%, and can even drop below 50%. Note that the situation where 100% is the largest duty cycle that prevents the first and second electrode assemblies from Tmax corresponds to the situation where the preferred channel remains active 100% of the time, which means that the non-preferred channel is never activated (until such time that changed conditions require the deactivation of the preferred channel to prevent the first and second electrode assemblies from exceeding Tmax).

In some embodiments the output voltages at the outputs Q1 and Q2 of the AC signal generator 20 are always linked, so that those output voltages always rise or fall in tandem. In these embodiments, if a single one of the electrode assemblies 10L/10R/10A/10P approaches the safety temperature threshold (e.g., 39° C.) before the other electrode assemblies 10, then the channel that includes that single (i.e., the hottest) electrode assembly would become a limiting factor from a thermal perspective. If the hottest electrode assembly happens to reside in the non-preferred channel, increasing the duty cycle of the preferred channel (as described herein) will therefore provide two benefits simultaneously. First, because the preferred channel will be operating for more time, the overall therapeutic effect of the alternating electric fields will increase. And second, because the nonpreferred channel will be operating at a lower duty cycle, the temperature at the hottest electrode assembly will drop, which will ameliorate the thermal limitation. If, on the other hand, the hottest electrode assembly happens to reside in the preferred channel, thermal considerations mandate that the duty cycle of the preferred channel can only be increased in these embodiments if the output voltages of both Q1 and Q2 are decreased.

In other embodiments, the output voltages at the outputs Q1 and Q2 of the AC signal generator 20 can be adjusted independently. In these embodiments, if the hottest electrode assembly happens to reside in the non-preferred channel, increasing the duty cycle of the preferred channel (as described herein) will provide the same two benefits described in the previous paragraph. But if the hottest electrode assembly happens to reside in the preferred channel, the AC signal generator 20 can reduce the output voltage of the preferred channel only (which will ameliorate the thermal limitation) and simultaneously increase the duty cycle of the preferred channel. And because the preferred channel will be operating for more time, the overall therapeutic effect of the alternating electric fields should increase.

Returning to FIG. 6, S60-S75 of FIG. 6 depict one approach for performing step (a) and step (b) with respective durations so that the duty cycle of step (a) is 85-100% (or, in some embodiments, 95-100%, 98-100%, or 100%) of the largest duty cycle that prevents the first and second electrode assemblies from exceeding a temperature threshold Tmax. This approach begins at S60, where the timing variables T1 and T2 are set to initial values (e.g., 1 second). Next, a first electric field E1 is induced in the ROI (using the preferred channel) for a duration T1 in step S62, and subsequently a second electric field E2 is induced in the ROI (using the non-preferred channel) for a duration T2 in step S64.

Next, in S66, tests are performed to ascertain whether any of the electrode assemblies are too hot. This can be accomplished by obtaining temperature readings from all of the electrode assemblies 10, and comparing those temperature readings to Tmax. If an electrode assembly that corresponds to the preferred channel (i.e., the first or second electrode assembly) is too hot, T1 is decreased in S70, after which processing returns to S62. (Decreasing T1 will reduce the amount of time that the preferred channel is active, which will reduce the temperatures of the first and second electrode assemblies.) Similarly, if an electrode assembly that corresponds to the non-preferred channel (i.e., the third or fourth electrode assembly) is too hot, T2 is decreased in S70, after which processing returns to S62. (Decreasing T2 will reduce the amount of time that the non-preferred channel is active, which will reduce the temperatures of the third and fourth electrode assemblies.)

Returning to S66, if none of the electrode assemblies are too hot, processing continues at S68, where a test is performed to ascertain whether the duty cycle of step (a) (which corresponds to S62) can be increased. This test can be implemented, for example, by obtaining temperature readings from the first and second electrode assemblies, and comparing those temperature readings to Tmax. If the duty cycle of step (a) cannot be increased without exceeding Tmax, processing returns to step S62 for another pass through the loop. If, other the other hand, the duty cycle of step (a) can be increased without exceeding Tmax, processing proceeds to step S75 where T1 is increased, after which processing returns to S62 for another pass through the loop. (Increasing T1 will increase the amount of the time that the preferred channel is active, which will increase the duty cycle.)

Optionally, in the embodiments that implement the processes depicted in FIG. 6, the AC signal generator 20 can have a third output (not shown in FIG. 3) that is configured to apply a third alternating voltage between a fifth electrode assembly and a sixth electrode assembly, and the at least one data input of the AC signal generator 20 is configured to accept data that represents temperatures of the fifth and sixth electrode assemblies. In these embodiments, the AC signal generator 20 is configured to repeatedly activate the first, second, and third outputs in an alternating sequence, and to adjust how long the first, second, and third outputs are activated based on data that represents temperatures of the first, second, third, fourth, fifth, and sixth electrode assemblies. The AC signal generator 20 is also configured to only activate the second and third outputs when the first output is deactivated.

Optionally, in these embodiments, the AC signal generator 20 is configured to only activate the third output when the first and second outputs are deactivated. In this case, there will be a three-tiered prioritization, with the first output having the highest priority, the second output having a mid-level priority, and the third output having the lowest priority. And the third output of the AC signal generator 20 will only be activated when neither the first output nor the second output can be activated based on thermal considerations.

Note that the approach described above in connection with FIG. 6 is not inconsistent with the approach described above in connection with FIG. 5, which means that it is possible to implement both of those approaches simultaneously. Thus, the system can be configured so that the preferred channel (i.e., the channel that provides the larger therapeutic effect) remains active for more time than the non-preferred channel and/or the preferred channel operates at (or near) the largest duty cycle that prevents the electrode assemblies from exceeding a temperature threshold Tmax. And prioritizing the preferred channel using either or both of these approaches can advantageously increase the overall therapeutic effect of the alternating electric fields.

Finally, it is important to note that the usage of the identifiers (a), (b), (c), (d), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying alternating electric fields to a region of interest, the apparatus comprising:
a signal generator having a first output, a second output, and at least one data input, wherein the first output is configured to apply a first 50 kHz-1 MHz alternating voltage between a first electrode assembly and a second electrode assembly, wherein the second output is configured to apply a second 50 kHz-1 MHz alternating voltage between a third electrode assembly and a fourth electrode assembly, and wherein the at least one data input is configured to accept data that represents temperatures of the first, second, third, and fourth electrode assemblies,
wherein the signal generator is configured to repeatedly activate the first and second outputs in an alternating sequence,
wherein the signal generator is configured to adjust how long the first and second outputs are activated based on data that represents temperatures of the first and second electrode assemblies, so that a duty cycle of the first output is 85-100% of a largest duty cycle that prevents the first and second electrode assemblies from exceeding a temperature threshold Tmax, and
wherein the signal generator is configured to only activate the second output when the first output is deactivated.

2. The apparatus of claim 1, wherein the duty cycle of the first output is 95-100% of the largest duty cycle that prevents the first and second electrode assemblies from exceeding the temperature threshold Tmax.

3. The apparatus of claim 1, further comprising:
the first electrode assembly;
the second electrode assembly;
the third electrode assembly; and
the fourth electrode assembly.

4. The apparatus of claim 1, wherein the signal generator has a third output configured to apply a third 50 kHz-1 MHz alternating voltage between a fifth electrode assembly and a sixth electrode assembly, and wherein the at least one data input is configured to accept data that represents temperatures of the fifth and sixth electrode assemblies,
wherein the signal generator is configured to repeatedly activate the first, second, and third outputs in an alternating sequence,
wherein the signal generator is configured to adjust how long the first, second, and third outputs are activated based on data that represents temperatures of the first, second, third, and fourth electrode assemblies, and
wherein the signal generator is configured to only activate the second and third outputs when the first output is deactivated.

5. The apparatus of claim 4, wherein the signal generator is configured to only activate the third output when the first and second outputs are deactivated.

6. A method of applying alternating electric fields to a region of interest, the method comprising:
(a) applying a 50 kHz-1 MHz alternating voltage between a first electrode assembly and a second electrode assembly positioned at respective first and second locations on opposite sides of the region of interest, wherein a first electric field is induced in the region of interest;
(b) applying a 50 kHz-1 MHz alternating voltage between a third electrode assembly and a fourth electrode assembly positioned at respective third and fourth locations on opposite sides of the region of interest, wherein a second electric field is induced in the region of interest; and
repeating step (a) and step (b) in an alternating sequence at least 100 times,
wherein step (a) and step (b) are performed with respective durations so that a duty cycle of step (a) is 85-100% of a largest duty cycle that prevents the first and second electrode assemblies from exceeding a temperature threshold Tmax.

7. The method of claim 6, wherein the duty cycle of step (a) is 95-100% of the largest duty cycle that prevents the first and second electrode assemblies from exceeding the temperature threshold Tmax.

8. The method of claim 6, further comprising:
running a plurality of simulations, each of which involves (a) positioning a plurality of model electrode assemblies at respective locations on a model of a subject's body and (b) predicting a therapeutic effect that a resulting electric field will provide within the region of interest when an alternating voltage is applied between the plurality of model electrode assemblies; and
selecting the first and second locations based on the plurality of simulations, wherein the first and second locations correspond to locations of the model electrode assemblies that provided the largest of all the predicted therapeutic effects.

9. The method of claim 6, further comprising:
running a plurality of simulations, each of which involves (a) positioning a plurality of model electrode assemblies at respective locations on a model of a subject's body and (b) predicting a therapeutic effect that a resulting electric field will provide within the region of interest when an alternating voltage is applied between the plurality of model electrode assemblies; and
selecting the first and second locations based on the plurality of simulations, wherein the first and second locations correspond to locations of the model electrode assemblies that provided a predicted therapeutic effect that was within the top 10 percent of all the predicted therapeutic effects.

10. The method of claim 9, further comprising selecting the third and fourth locations based on the selected first and second locations.

11. A method of applying alternating electric fields to a region of interest, the method comprising:
(a) applying a 50 kHz-1 MHz alternating voltage between a first electrode assembly and a second electrode assembly positioned at respective first and second locations on opposite sides of the region of interest, wherein a first electric field is induced in the region of interest;
(b) applying a 50 kHz-1 MHz alternating voltage between a third electrode assembly and a fourth electrode assembly positioned at respective third and fourth locations on opposite sides of the region of interest, wherein a second electric field is induced in the region of interest; and
repeating step (a) and step (b) in an alternating sequence at least 100 times,
wherein step (a) and step (b) are performed with respective durations so that either
(i) in the aggregate, step (a) is performed for more time than step (b) if a determination has previously been made that the first electric field will provide a larger therapeutic effect than the second electric field or
(ii) in the aggregate, step (b) is performed for more time than step (a) if a determination has previously been made that the second electric field will provide a larger therapeutic effect than the first electric field.

12. The method of claim 11, further comprising:
(c) applying a 50 kHz-1 MHz alternating voltage between a fifth electrode assembly and a sixth electrode assembly positioned at respective fifth and sixth locations on opposite sides of the region of interest, wherein a third electric field is induced in the region of interest,
wherein step (a), step (b), and step (c) are repeated in an alternating sequence at least 100 times, and
wherein step (a) is performed for more time than step (b) and for more time than step (c)
if a determination has previously been made that the first electric field will provide a larger therapeutic effect than the second electric field and the third electric field.

13. The method of claim 11, further comprising determining whether the first electric field or the second electric field will provide a larger therapeutic effect, wherein the determining occurs before the repeating.

14. The method of claim 11, further comprising:
simulating the first electric field in the region of interest based on (i) the respective locations of the first and second electrode assemblies and (ii) characteristics of tissue located between the first and second electrode assemblies;
simulating the second electric field in the region of interest based on (i) the respective locations of the third and fourth electrode assemblies and (ii) characteristics of tissue located between the third and fourth electrode assemblies; and
determining whether the first electric field or the second electric field will provide a larger therapeutic effect by comparing the simulation of the first electric field to the simulation of the second electric field,
wherein the determining occurs before the repeating.

15. The method of claim 14, wherein the comparing of the simulations comprises comparing field strengths within the region of interest for the first electric field to field strengths within the region of interest for the second electric field.

16. The method of claim 14, wherein the comparing of the simulations comprises comparing power densities within the region of interest for the first electric field to power densities within the region of interest for the second electric field.

17. The method of claim 11, wherein if the determination has previously been made that the first electric field will provide a larger therapeutic effect than the second electric field, step (a) and step (b) are performed with respective durations so that, in the aggregate, step (a) is performed for at least 10% more time than step (b).

18. The method of claim 11, wherein if the determination has previously been made that the first electric field will provide a larger therapeutic effect than the second electric field, step (a) and step (b) are performed with respective durations so that, in the aggregate, step (a) is performed for at least 25% more time than step (b).

19. The method of claim 11, wherein if the determination has previously been made that the first electric field will provide a larger therapeutic effect than the second electric field, step (a) and step (b) are performed with respective durations so that the time spent performing step (a) is maximized to an extent that is possible in view of thermal considerations, and so that step (b) is performed as needed to prevent the first electrode assembly and the second electrode assembly from exceeding a temperature threshold.

20. The method of claim 11, wherein the first electric field and the second electric field are perpendicular, ±30°, within the region of interest.
